(19) United States
(12) Patent Application Publication
(10) Pub. No.: US 2006/0120999 A1
(43) Pub. Date: Jun. 8, 2006

(54) Protein and peptide expression for passive immunity

(76) Inventors: Arun Kumar Dhar, San Diego, CA (US); F.C. Thomas Allnutt, Glenwood, MD (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/525,364

(22) PCT Filed: Sep. 15, 2003
(86) PCT No.: PCT/US03/28868

§ 371 Date: Sep. 22, 2005

Related U.S. Application Data
(60) Provisional application No. 60/410,818, filed on Sep. 16, 2002.

(51) Int. Cl. A61K 48/00

(52) U.S. Cl. 424/93.2

(57) ABSTRACT

This invention relates to feeds, feed supplements, and methods for their use that provide disease controlling properties to humans, and terrestrial and aquatic animals. These methods and compositions have both acute and chronic applications. The chronic applications relate to the health of organisms that have primitive or poorly developed immune systems.

This application claims the priority of provisional application 60/410,818, which was filed in the United States Patent and Trademark Office on Sep. 16, 2002, the disclosure of which is herein incorporated by reference.

### BACKGROUND OF THE INVENTION

Passive immunity is the delivery of immune function directly to an animal without the need for an immune response. It is commonly referring to the delivery of antibodies produced in one organism to a naïve organism in order to provide protection from a specific disease or symptom (Zhang et al. 1989; Lorenzen et al. 1990; Lee et al. 1997). An analogous approach is the delivery of a compound or compounds that prevent binding of the infectious agent to its site of infection, either directly or by competition for the binding site. Many drugs are based on this type of interaction.

Viral diseases cause a huge amount of damage in humans, terrestrial, and aquatic animals. Organisms that have primitive or poorly developed immune systems are especially susceptible to viral disease. Crustaceans, such as shrimp, do not have adaptive immunity. Instead they rely on the innate immune response. Although several immune genes involved in bacterial and fungal immunity in invertebrates have been well characterized, very few of the immune genes that are involved in viral pathogenesis are known from shrimp or any other invertebrate, so far.

Viral diseases cause a huge amount of economic loss in crustacean aquaculture. A number of viruses are important to shrimp aquaculture and cause billions of dollars worth of damage annually with virtually no therapeutic treatment available to combat this problem. Yellow head virus (YHV), Taura syndrome virus (TSV), Infectious hypodermal and hematopoietic necrosis virus (IHHNV), and White spot syndrome virus (WSSV; also known as White spot virus, WSV and White spot bacilloform virus, WSBV) have caused pandemics that have affected global penaeid shrimp farming (Lightner 2002).

White spot disease, caused by the white spot virus (WSSV), is currently the most important viral disease of cultured penaeid shrimp (Penaeus sp.) worldwide. WSV has a bacilliform enveloped morphology, and the genome contains a circular double stranded DNA of ˜292 to 305 Kb (van Hulten et al. 2001; Yang et al. 2001). The SDS-PAGE analysis of purified WSV showed four major polypeptides with estimated molecular masses of 28 kDa (VP28), 26 kDa (VP26), 24 kDa (VP24), and 19 kDa (VP19) (van Hulten et al. 2000). Out of these four proteins, VP26 and VP24 are associated with the nucleocapsids, whereas VP28 and VP19 remain with the envelope (van Hulten et al. 2000). Although considerable progress has been made in the detection and molecular characterization of WSV in recent years, efforts to develop therapeutics to prevent white spot disease have not been developed.

Molecular methods have been developed to express proteins in yeast (Elledge et al. 1991; McGonigal et al. 1998; Cereghino and Cregg 1999; Cregg et al. 2000), bacteria (Iyer et al. 2002), plants (Mason et al. 1992; Kapusta et al. 1999), fungi (Oyama et al. 2002), and algae (Lapidot et al. 2002; Shapira et al. 2002; Ton et al. 2002). Many of the hosts for the previous systems for protein expression have been used in feeds for terrestrial animals and aquatic animals.

Oral delivery of drugs and vaccines is common (Cho and Howard 1999; Tacket et al. 2000; Bootland et al. 2002). Expression of proteins, including viral proteins, in bacteria, yeast, fungi, plants, animals, and algae as well as tissue cultures thereof is also previously described.

There is a need for new methods to combat viral disease in humans, terrestrial animals, and aquatic animals. The need is extreme in cases where the immune system is primitive or poorly developed. One example is crustaceans, where there is a primitive immune system relying not on antibodies but on innate immunity and production of lectins.

### SUMMARY OF THE INVENTION

The invention provides a method of protecting an animal from disease by producing, in a transformed host cell, a disease-related protein or peptide from an agent that causes disease, and delivering the protein or peptide to an animal suspected of being infected by the agent. According to this method, delivery of the protein inhibits or retards binding of the agent that causes the disease in one or more cells of the animal. The agent that causes the disease can be, e.g., a virus, a bacterium, or a prion.

According to this method, the disease-related protein or peptide is produced by transforming a host cell with a nucleic acid encoding the disease-related protein to form a transformed cell. The host cell can be chosen from bacteria, algae, yeast, fungi, insects, animals, plants, and tissue cultures of any of these.

According to this method, the disease-related protein or peptide can be a viral protein or peptide. For example, this viral protein or peptide can comprise one or more segments of white spot syndrome virus. The viral protein or peptide can comprise one or more segments of white spot syndrome viral protein VP26, VP28, VP19, and VP24.

The invention also provides a feed that is supplemented with a recombinant protein or peptide that competes with a disease-causing agent to reduce or alleviate a disease state. The recombinant protein or peptide in the feed can comprise at least a portion of a viral protein. The recombinant protein or peptide in the feed can comprise white spot syndrome virus sequences, including one or more one or more of VP24, VP28, VP26, and VP19.

The invention further provides a feed additive comprising a recombinant protein or peptide that competes with a disease-causing agent to reduce or alleviate a disease state. This feed additive can be fed to an animal as whole cells or broken cells. It can also be fed to an animal as purified or semi-purified protein, or encapsulated versions of these.

The recombinant protein or peptide of the feed additive can comprise at least a portion of a viral protein. This recombinant protein or peptide can comprise white spot syndrome virus sequences, including one or more one or more of VP24, VP28, VP26, and VP19.

The invention yet further provides a method of protecting an animal from disease, comprising producing a protein or peptide capable of binding a disease-causing agent in a transformed host cell, and delivering the protein or peptide to an animal suspected of being infected by the agent. According to this method, delivery of the protein inhibits or retards binding of the agent that causes the disease in one or more cells of the animal.

The invention provides a feed comprising a recombinant protein or peptide capable of binding a disease-causing agent and reducing or alleviating a disease state. It also provides a feed additive comprising a protein or peptide capable of binding to a disease-causing agent. This feed additive can be fed as whole cells or broken cells. It can also be fed to an animal as purified or semi-purified protein, or encapsulated versions of these.

### DETAILED DESCRIPTION OF THE INVENTION

Definitions

In describing the present invention, the following terminology is used in accordance with the definitions set out below.

“Passive immunity” is defined here as delivery of either antibodies or proteins that deliver protection from infection, either by binding to the virus or to its receptor or blocking the mode of entry into the animal.

A “primitive immune system” is defined as a system lacking the production of specific antibodies in response to the presence of antigen or having a weak antigen-mediated immune response. This is found in several classes of organisms including but not limited to invertebrates, crustaceans, annelids, nematodes, rotifers, mollusks, echinoderms, insects, chelicerates, protists, ascidians, sponges, and corals.

A “target animal” is defined as an animal that is threatened by a disease-causing element.

A “feed” is defined as a preparation providing nutritional value to any animal, including but not limited to terrestrial animals (humans, cattle, horses, pigs, sheep, goats, poultry) and aquatic animals (fish, shrimp, lobsters, crawfish, mollusks, sponges, jellyfish).

A “feed additive” is anything that is added to an animal's feed, regardless of nutritional value.

### EMBODIMENTS OF THE INVENTION

In one aspect, the invention provides a viral protein in yeast, bacteria, plants, fungi, animals, insects, and algae, as well as tissue/cell cultures of these systems. The protein can be provided to animals to provide protection from viral infection. Accordingly, the invention provides a method of producing the protein.

In another aspect, the invention provides a fusion protein containing a viral protein portion. The fusion protein can be orally provided to animals to provide protection from viral disease. Accordingly, the invention provides a method of producing the fusion protein.

In yet another aspect, the invention protects an animal from viral infection by blocking binding of virus to its receptors in the gut. This aspect is achieved by providing competing viral protein(s) and/or peptides administered orally.

In yet a further aspect, the invention protects an animal from viral infection by blocking binding of virus to its receptors in the gut. This aspect is achieved by providing binding moieties of its receptor(s) on the gut.

These and other aspects of the invention are provided by one or more of the following embodiments.

In one embodiment of the invention, a method of protecting an animal from viral infection is provided. The method comprises the steps of:

producing a viral protein and/or peptide, such as a capsid or envelope protein, in a foreign host expression system, such as in a yeast, fungus, bacterium, alga, insect, animal, or plant, or alternatively in tissue/cell cultures of these systems,

processing the biomass containing the viral protein into a feed or feed supplement with minimal purification, and

providing the biomass to the animal to deliver the viral protein in an amount up to 0.01 to 50% of the total animal feed content,

wherein the presence of the viral protein competes with live virus inside the animal to prevent infection.

In another embodiment of the invention, a method of protecting an animal from a viral infection is provided. The method comprises the steps of:

producing a receptor or receptor moiety to which a virus attaches for infection in a foreign host expression system, such as in a yeast, fungus, bacterium, algal, insect, animal, or plant tissue cultures thereof,

processing the biomass containing the virus-binding receptor into a feed or feed supplement with minimal purification, and

providing the processed biomass to the animal to deliver the virus-binding domain in an amount up to 5% of the total animal feed protein content,

wherein the presence of the virus-binding domain prevents live virus binding and infection in the animal.

This invention provides a rapid response to a viral disease threat. It can also be applied to other types of diseases caused by bacteria, prions, DNA, protists, and other disease causing organisms or factors.

In organisms without well developed immune systems, the invention provides methods for treating both acute and chronic disease via delivery of preformed virus proteins, virus binding domains, receptors for the virus, domains from the receptor that bind virus, or similar functional units that will tie up either free virus or block binding of the virus to its receptor through competition. These can be delivered chronically, or for acute treatment of an infection.

In organisms with well-developed immune systems, the invention provides a first response method to retard the onset an acute infection threat until the immune response can be mounted.

The first approach involves expression in a number of different systems (e.g., bacterial, plant, algal, fungal, insect, and yeast and tissue cultures thereof) of a viral protein or proteins. These proteins can be the whole protein or just the domain, which recognizes the virus' receptor on the mucosal lining. These are then fed to the target animal either as whole cells or broken cells or purified or partially purified protein to compete with the virus for binding to the mucosal lining. Such competition will retard or prevent viral infection.

The second approach involves expression in a number of different systems (e.g., bacterial, plant, algal, fungal, insect, and yeast and tissue cultures thereof) of the receptor or binding site of the receptor for the virus of interest. These receptors are limited in only the binding affinity to the virus of interest and can be truncated or modified as needed. The receptor mimetic would bind to the virus to compete with the mucosal receptor and inactivate the virus, thereby preventing infection.

A third approach to preventing the uptake of live WSSV by the shrimp is to provide a high concentration of a viral binding protein in a whole or lysed recombinant cell (or semipurified preparation) such that most, if not all, of the live viral particles will bind to the mimetic and not to the shrimp viral binding site, thereby minimizing infectivity.

Dhar and colleagues have studied patterns of differentially expressed genes in shrimp following infection by WSSV (Dhar et al. 2001; Astrofsky et al. 2002) and found a gene coding for a lipopolysaccharide/beta-glucan binding protein (LGBP) to be one of several genes that are up regulated (Roux et al. 2002). Recognition proteins, such as LGBP, play a key role in the non-specific immune response (NSIR) of insects and crustaceans. LGBP may represent the endogenous viral binding protein in shrimp that contains 1,352 base pairs coding for a polypeptide of 376 amino acids in length (Roux et al. 2002). A protein of this size is unlikely to cross the mucosal membrane and therefore may represent the endogenous viral binding protein known to activate the prophenol oxidase cascade. Consequently, if such a protein is delivered in a feed or feed supplement and exposed to the mucosal tissues, it may specifically bind the virus (similar to an antibody), preventing it from binding to the endogenous binding site in the mucosal tissues and thereby preventing the initiation of the infection process. It may also be desirable to reduce the size of the LGBP by expressing only the LOBP binding domain (i.e., the region that contains the B-1,3-linkage of polysaccharide and the arginine-glycine-aspartic acid (RGD) motifs as indicated by consensus sequences with homologous proteins. A truncated protein may have similar binding affinity, but be more resistant to cleavage by endogenous proteinases.

### EXAMPLES

The following examples are provide for exemplification purposes only and are not intended to limit the scope of the invention.

### Example 1

### Production of Recombinant White Spot Virus Proteins VP19 in a Yeast Expression System

The gene for WSSV protein VP19 is available from the GenBank database (AF369029). Primers are designed to amplify the entire VP19 protein. PCR/RT-PCR is performed to amplify the entire gene as well as the hydrophilic domains of VP19 gene using standard methods (Sambrook et al. 1989). Cloning of full-length VP19 gene using the pYES2-DES52 Saccharomyces cerevisiae expression system (Invitrogen, Inc.) is carried out with GAL1 promoter applied for separate expression of the two viral genes simultaneously under galactose induction. The transformants are screened by PCR with sequencing of the positive clones to ensure their identity with the original sequence. Western blot detection methods are used to validate production of protein using standard methods (Sambrook et al. 1989).

### Example 2

### Production of Recombinant White Spot Virus Proteins VP28 and VP26 in a Yeast Expression System

The genes for WSSV proteins VP26, and VP28 DNA are available from the GenBank database (AF173992, AF173993). Primers are designed to amplify the entire VP26 and VP28 proteins. PCR/RT-PCR is performed to amplify the entire gene as well as the hydrophilic domains of VP26 and VP28 genes using standard methods (Sambrook et al. 1989). Cloning of full-length VP26 and VP28 genes using Saccharomyces cerevisiae expression system pESC (Stratagene) is carried out with Gal1 and Gal10 promoters applied for separate expression of the two viral genes simultaneously under galactose induction. The transformants are screened by PCR with sequencing of the positive clones to ensure their identity with the original sequence. Western blot detection methods are used to validate production of protein using standard methods (Sambrook et al. 1989).

### Example 3

### Method for Protection of Shrimp from WSSV Infection

Shrimp are fed recombinant Saccharomyces cerevisiae containing proteins derived from WSV coat protein genes (as in Examples 1 and 2); these proteins appear to block the viral receptors needed for WSV infection to provide a passive immunity to the animals and provide some protection from WSSV disease. The yeast are provided in either whole or broken form directly to the fish in a microbound format in beads composed of alginate and starch in a polymeric form. Alternative microbound forms are available such as polyactide (Bootland et al. 2002), carrageen, alginate, and chiotsan. Attractants can be added to make the beads more easily consumed by the target species (in the case of shrimp, krill meal would be a good alternative). A challenge with the WSSV will result in increased survivability in response to viral infection in shrimp fed the recombinant yeast.

### Example 4

### Production Truncated Recombinant White Spot Virus Proteins VP28 and VP26 in a Yeast Expression System

The genes for WSSV proteins VP26, and VP28 DNA are available from the GenBank database (AF173992, AF173993) as in Example 2. Using the hydrophilicity profile of VP26 and VP28 proteins the hydrophilic domains are identified. PCR/RT-PCR is performed to clone the truncated VP26 and VP28 using the pESC Saccharomyces cerevisiae expression system (Stratgene), and standard methods (Sambrook et al. 1989). This is followed by screening of recombinant clones by PCR and sequencing the clones to ensure their identity with the original sequence. Recombinant protein production is assayed by Western blot analysis using WSV VP26 and VP28 antibodies. Antibodies are available for VP26 (DiagXotics, Inc., CT) and are made as polyclonals by custom contract with Immuno-Precise Antibodies (Victoria, Canada).

### Example 5

### Production of LGBP in Yeast Expression System

Dhar and colleagues have cloned and sequenced shrimp LGBP gene and the nucleotide sequence is available in the GenBank database (AF473579) (Roux et al. 2002). LGBP is a known elicitor of prophenoloxidase (ProPO) cascade in arthropods. The ProPO cascade is one of the well-characterized defense mechanisms of invertebrates. The entire LGBP gene encoding an open reading frame of 326 amino acids is cloned in a yeast expression system (pYES2.1 TOPO TA expression system, Invitrogen Inc.). The truncated LGBP gene that contains the putative binding site for β-1,3 linkage for polysaccharide and the cell attachment binding domain (RGD motif) is amplified by RT-PCR and cloned into the yeast expression cassette (pYES2.1 TOPO TA expression system, Invitrogen Inc.). Recombinant protein is made out of the binding region and expressed in yeast expression system as in Example 1.

### Example 6

### Protection of Shrimp From WSSV Infection Using Recombinant LGBP of Example 5

Yeast from Example 5 are mixed with the feed either in a microbound format (as in Example 3) or directly in cold extruded feeds. Feeds are then provided to shrimp and protection is provided from infection by WSSV by the binding of LGBP to WSSV and the activation of the ProPO cascade.

### Example 7

### Production of Recombinant IPNV VP2 Protein in Bacteria

Genes for the VP2 capsid protein of infectious pancreatic necrosis virus, a fish virus, are cloned according to existing literature (Yao and Vakharia 1998). The gene is cloned into pTrcHis vector (Invitrogen), a protein expression vector for Escherichia coli. The protein is expressed behind the Trc promoter (a version of the Trp promoter) and expressed in the cell. Whole cells are harvested that contain the gene on induction by either IPTG (isopropyl-1-β-D-galactoside) or other inducer of the Trc promoter. Production of the recombinant protein is validated by western analysis using standard methods and antibody for Immuno-Precise Antibodies produced to IPNV isolated by our laboratory (Sambrook et al. 1989).

### Example 8

### Protection of Fish From IPNV Infection Using Recombinant IPNV VP2 Protein Expressed in Bacteria

Recombinant bacteria from Example 7 are fed, either formulated, encapsulated, or directly, to fish (such as hybrid striped bass or salmon) at a final recombinant protein concentration of less than 100 mg/kg. The VP2 competes with virus for binding to the receptors within the gut of the fish to provide protection from IPNV infection.

### Example 9

### Protection of Shrimp From WSSV Infection by Expression of a Ligand Binding Domain of the Virus

Recombinant green algae that are expressing the receptor for WSSV (Chlorella vulgaris) are produced using established methods (Choi et al. 2000). The cells are grown either photosynthetically in enclosed photobioreactors (Rebolloso-Fuentes et al. 2001; Lebeau et al. 2002) or in traditional fermentors (Running et al. 1994). Recombinant cells are fed directly to shrimp experiencing an outbreak of WSSV to prevent binding of the virus to its receptor, thereby preventing disease.

### Example 10

### Acute Protection of Animals that have an Highly Developed Immune System

Animals such as humans, terrestrial agricultural animals (e.g., cows, horses, sheep, swine, rabbits, goats), aquatic animals (e.g., fish), and pets (e.g., dogs, cats) that have a higher immune system can be protected in a manner analogous to that for the primitive immune system animals described in Example 9, or Examples 1-3, as protection from initial infection during an outbreak prior to the induction of antibody production. Virus receptors or viral proteins that mediate binding to the receptor can be provided as a first response to an infection to protect the animal while the body begins to respond with the immune system.

### Example 11

### Expression of WSSV Proteins in a Green Alga, Chlorella vulgaris

The genes coding for VP19 and VP28 are ligated into the pCNR/HUP vector at a site downstream and under control of the NR-promoter (nitrate reductase) to generate the transformation plasmids pCNR/HUP/VP19 and pCNR/HUP/VP28. These plasmids are used to transform HUP⁽⁻⁾ Chlorella (hexose uptake minus mutants) using the particle bombardment procedure (Biolistics®) and transformants are selected by growth in the dark on glucose (Allnutt et al. 2000). Transformed colonies are subcultured and tested for the production of the presence of VP19 and VP28 by Western blot analysis using antibodies and standard techniques (Sambrook et al. 1989).

The binding affinities of VP19 and VP28 relative to intact WSSV are determined using a standard competitive binding assay (Chan and Perlstein 1987) and anti-WSSV antibody coated microplates. WSSV labeled with a fluorescent marker (e.g., phycocyanin) is added to each well of the microplate along with serial dilutions of the extracts from the VP19 or VP28 producing NC's. The titration curve of the fluorescence provides an estimate of the binding affinity of the viral mimetic relative to the virus itself. Truncated versions of the virus proteins can also be made that deliver similar binding affinities and used for provision of passive protection against WSSV infection.

The recombinant C vulgaris cells expressing the WSSV virus proteins are fed directly to shrimp or supplied as a component in the feeds as described in Examples 3 and 6.

### Example 12

### Production of Recombinant TSV Capsid Proteins in a Yeast Expression System

Taura syndrome disease, caused by Taura syndrome virus (TSV), is one of the most important viral diseases of penaeid shrimp in the Western Hemisphere (Hasson et al. 1995; Brock 1997). Since the initial report of the disease in 1992 in Ecuador, the disease has been reported in 13 different countries in the Americas, and recently in Taiwan (Hasson et al. 1999; Tu et al. 1999). Taura syndrome disease has caused catastrophic losses in farmed shrimp in the Americas (Brock et al. 1997).

TSV particles are non-enveloped, spherical, 31-32 nm diameter and the capsid is composed of three major proteins of 55, 40 and 24 kDa and one minor protein of 58 kDa (Bonami et al. 1997). The viral genome contains linear positive sense single stranded RNA of approx. 10 kb which is polyadenylated at the 3′-end (Bonami et al. 1997). Dhar and colleagues cloned and sequenced a 3278 bp cDNA representing the 3′ end of the TSV genome (Accession number AF277378, (Robles-Sikisaka et al. 2001)). Sequence analyses revealed that the TSV capsid protein genes are located at the 3′-end of the genome, and phylogenetic analysis showed that the genome organization of TSV of shrimp is similar to insect picornaviruses (Robles-Sikisaka et al. 2001). Recently, the entire genome of TSV has been sequenced (Accession number AF277675, (Mari et al. 2002)). The genome of TSV was found to be 10,205 nucleotides in length, containing two open reading frames (ORFs). The non-structural genes (helicase, protease, and RNA dependent RNA polymerase) are located at the 5′ end and the structural genes (capsid proteins) are located at the 3′ end of the genome (Mari et al. 2002). TSV genome is transcribed as a single transcript of approx. 10 kb size, and the coat proteins are not expressed as subgenomic RNA (Robles-Sikisaka et al. 2001). Therefore, it appears that the TSV transcript is translated into a large polypeptide that undergoes a proteolytic cleavage by TSV encoding protease to make functional proteins, as seen in other picornaviruses.

The nucleotide sequence of TSV capsid protein genes is available in the GenBank database (Accession number AF277378, (Robles-Sikisaka et al. 2001)). Primers will be designed flanking the entire TSV capsid ORF that encodes four capsid proteins. Primers are designed to amplify the TSV protease gene (Accession number AF277675, (Mari et al. 2002)). RT-PCR is performed to amplify the capsid and the protease genes following the published protocol (Robles-Sikisaka et al. 2001). The amplified cDNA is cloned into a Saccharomyces cerevisiae expression vector pESC (Stratagene, Inc.). The expression of the capsid and the protease genes is carried out with Gal1 and Gal10 promoters simultaneously under galactose induction. The transformants are screened by PCR, and the positive clones sequenced to ensure the identity of the TSV genes with the original sequence. The expression of the TSV capsid proteins are confirmed by Western blot analysis using standard methods (Sambrook et al. 1989). The recombinant TSV protease cleaves the recombinant TSV capsid polypeptide into functional proteins with expected size, i.e. 58, 55, 40, and 24 kDa. These recombinant proteins self assemble in vitro to form Taura syndrome virus like particle (TS-VLP). Such a TSV ghost protein shell, that lacks an infectious TSV RNA, is used as an additive into shrimp diet.

### Example 13

### Production of Recombinant TSV Capsid Proteins Using a Bacterial Expression System

TSV capsid protein gene sequence is available in the GenBank database (Accession number AF277378, (Robles-Sikisaka et al. 2001)). Primers are designed flanking each of the four capsid proteins separately. RT-PCR is performed to amplify each capsid gene following published protocol (Robles-Sikisaka et al. 2001). Each amplified cDNA is cloned separately into a bacterial expression vector pQE-30UA (Qiagen, Inc.). The transformants are screened by PCR, and the positive clones sequenced to confirm the identity of the cloned TSV genes. The expression of the capsid proteins are carried out in Escherichia coli ML15 cell line under IPTG induction. The expression of the TSV capsid proteins is confirmed by Western blot analysis using standard methods (Sambrook et al. 1989).

### Example 14

### Protection of Shrimp From TSV Infection by Using Recombinant TSV Protein Expressed in Yeast

Shrimp are fed recombinant Saccharomyces cerevisiae containing TS-VLP. These VLPs block the viral receptors needed for TSV infection, and thus provide a passive immunity to the animals against TSV. The yeast are incorporated into shrimp diet either as a whole or broken form directly in a microbound format in beads composed of alginate and starch in a polymeric form. Alternative microbound forms are available such as polyactide (Bootland et al. 2002), carrageen, alginate, and chiotsan. Attractants, such as krill meal, are added to make the beads more palatable to shrimp. Shrimp are fed with a diet containing TS-VLP before challenging with infectious TSV. Shrimp fed with a diet containing TS-VLP have increased survivability against TSV infection.

### Example 15

### Protection of Shrimp From TSV Infection by Using Recombinant TSV Protein Expressed in Bacteria

Recombinant E. coli expressing four different TSV coat proteins are mixed together before mixing with a commercial shrimp diet. These proteins block the TSV receptors needed to initiate infection, and thus provide a passive immunity to the animals against TSV. The E. coli are incorporated in shrimp diet either as a whole or broken form directly in a microbound format in beads composed of alginate and starch in a polymeric form. Shrimp are fed with diet containing TSV recombinant proteins before challenging with infectious TSV. Shrimp fed with diet containing TSV recombinant proteins have increased survivability against TSV infection.

### Example 16

### Protection of Shrimp from TSV Infection by Expressing TSV Capsid Protein Genes in Algae, and Using Algae as a Shrimp Feed Additive

Recombinant green algae (Chlorella vulgaris) expressing four individual TSV capsid proteins are produced using established methods (Choi et al. 2000). The cells are grown either photosynthetically in enclosed photobioreactors (Rebolloso-Fuentes et al. 2001; Lebeau et al. 2002) or in traditional fermentors (Running et al. 1994). Recombinant cells expressing four different TSV capsid proteins are mixed and fed directly to shrimp followed by a challenge with infectious TSV. Shrimp fed with algae expressing TSV capsid protein will be able to prevent TSV infection.

### REFERENCES

Allnutt F, Kyle D, Grossman A, Apt K (2000) Methods and tools for transformation of eukaryotic algae. In: U.S. Pat. No. 6,027,900. Carnegie Institution of Washington; Martek Biosciences Corporation
Astrofsky K M, Roux M M, Klimpel K R, Fox J G, Dhar A K (2002) Isolation of differentially expressed genes from white spot virus (WSV) infected Pacific blue shrimp (Penaeus stylirostris). Arch Virol 147:1799-1812
Bonami J R, Hasson K W, Mari J, Poulos B T, Lightner D V (1997) Taura syndrome of marine penaeid shrimp: characterization of the viral agent. J Gen Virol 78 (Pt 2):313-319
Bootland L, Lizama M, Lin W, Salonius K (2002) Oral immunization of salmonids with biodegradable microparticle-based vaccines. In: Harrington K (ed) 4th Intl Symp Aquatic Animal Health, New Orleans, p 228
Brock J A (1997) Special topic review: Taura syndrome, a disease important to shrimp farms in the Americas. Wld J Micro Biotech 13:415-418
Brock J A, Gose R, Lightner D, Hasson K (1997) Recent developments and an overview of Taura Syndrome of farmed shrimp in the Americas. In: Flegel T, MacRae I (eds) Diseases in Asian Aquaculture in the Americas. Asian Fisheries Society, Manila, Phillippines, pp 275-284
Cereghino G P, Cregg J M (1999) Applications of yeast in biotechnology: protein production and genetic analysis. Curr Opin Biotechnol 10:422-427
Chan D, Perlstein M (1987) Immunoassay: A practical guide. Academic Press, New York
Cho H W, Howard C R (1999) Antibody responses in humans to an inactivated hantavirus vaccine (Hantavax). Vaccine 17:2569-2575
Choi T-J, Kim Y-T, Kim D-H (2000) Biosynthesis of foreign proteins using transformed microalgae. In: World Intellectual Property Organization. ALGenetech
Cregg J M, Cereghino J L, Shi J, Higgins D R (2000) Recombinant protein expression in Pichia pastoris. Mol Biotechnol 16:23-52
Dhar A K, Roux M M, Klimpel K R (2001) Detection and quantification of infectious hypodermal and hematopoietic necrosis virus and white spot virus in shrimp using real-time quantitative PCR and SYBR Green chemistry. J Clin Microbiol 39:2835-2845
Elledge S, Mulligan J, Ramer S, Spottswood M, Davis R (1991) Yes: A multifunctional cDNA expression vector for the isolation of genes by complementation of yeast and Escherichia coli mutations. Proc. Natl. Acad. Sci, USA 88:1731-1735
Hasson K et al. (1995) Taura syndrome in Penaeus vannamei: demonstation of a viral etiology. Dis Aquat Organ 23:115-126
Hasson K W et al. (1999) The geographic distribution of Taura syndrome virus (TSV) in the Americas: Determination by histopathology and in situ hybridization using TSV-specific cDNA probes. Aquacult 171:13-26
Iyer R B, Wang J, Bachas L G (2002) Cloning, expression, and characterization of the gsdA gene encoding thermophilic glucose-6-phosphate dehydrogenase from Aquifex aeolicus. Extremophiles 6:283-289
Kapusta J et al. (1999) A plant-derived edible vaccine against hepatitis B virus. Faseb J 13:1796-1799
Lapidot M, Raveh D, Sivan A, Arad S M, Shapira M (2002) Stable chloroplast transformation of the unicellular red alga Porphyridium species. Plant Physiol 129:7-12
Lebeau T, Gaudin P, Moan R, Robert J M (2002) A new photobioreactor for continuous marennin production with a marine diatom: influence of the light intensity and the immobilised-cell matrix (alginate beads or agar layer). Appl Microbiol Biotechnol 59:153-159
Lee K, Liu P, Kou G, Chen S (1997) Passive immunization of the tiger prawn, Penaeus mondon, using rabbit antisera to Vibrio harveyi. Lett Appl Microbiol 25:34-37
Lightner D (2002) Introduction of non-indigenous shrimp viruses and their potential impact on farmed and native wild shrimp populations. In: Harrington K (ed) 4th Intl Symp Aquatic Animal Health, New Orleans, p 86
Lorenzen N, Olesen N, Jorgensen P (1990) Neutralization of Egtved virus pathogenicity to cell cultures and fish by monoclonal antibodies to the viral G protein. J Gen Virol 71:561-567
Mari J, Poulos B T, Lightner D V, Bonami J R (2002) Shrimp Taura syndrome virus: genomic characterization and similarity with members of the genus Cricket paralysis-like viruses. J Gen Virol 83:915-926
Mason H S, Lam D M, Arntzen C J (1992) Expression of hepatitis B surface antigen in transgenic plants. Proc Natl Acad Sci USA 89:11745-11749
McGonigal T, Bodelle P, Schopp C, Sarthy A (1998) Construction of a sorbitol-based vector for expression of heterologous proteins in Saccharomyces cerevisiae. Appl Environ Microbiol 64
Oyama S, Yamagata Y, Abe K, Nakajima T (2002) Cloning and expression of an endo-1,6-beta-D-glucanase gene (neg1) from Neurospora crassa. Biosci Biotechnol Biochem 66:1378-1381
Rebolloso-Fuentes M M, Navarro-Perez A, Garcia-Camacho F, Ramos-Miras J J, Guil-Guerrero J L (2001) Biomass nutrient profiles of the microalga Nannochloropsis. J Agric Food Chem 49:2966-2972
Robles-Sikisaka R, Garcia D K, Klimpel K R, Dhar A K (2001) Nucleotide sequence of 3′-end of the genome of Taura syndrome virus of shrimp suggests that it is related to insect picornaviruses. Arch Virol 146:941-952
Roux M M, Pain A, Klimpel K R, Dhar A K (2002) The lipopolysaccharide and beta-1,3-glucan binding protein gene is upregulated in white spot virus-infected shrimp (Penaeus stylirostris). J Virol 76:7140-7149
Running J, Huss R, Olson R (1994) Heterotrophic production of ascorbic acid by microalgae. J Appl Phycol 6:99-104
Sambrook J, Fritsch E, Maniatis T (1989) Molecular Cloning: A laboratory manual, 2 edn. Cold Spring Harbor Press, Cold Spring Harbor
Shapira M, Arad S M, Lapidot M, Raveh D, Sivan A (2002) Stable chloroplast transformation of the unicellular red alga Porphyridium species. Plant Physiol 129:7-12
Tacket C O, Mason H S, Losonsky G, Estes M K, Levine M M, Arntzen C J (2000) Human immune responses to a novel norwalk virus vaccine delivered in transgenic potatoes. J Infect Dis 182:302-305.
Ton G, Ni K, Cohen A, Mayfield S (2002) Construction of the anti-cocaine Fab genes for expression in the unicellular green alga Chlamydomonas reinhardtii. FASEB J 16:A542
Tu C et al. (1999) Taura syndrome in Pacific white shrimp Penaeus vannamei cultured in Taiwan. Dis Aquat Organ 38:159-161
van Hulten M C et al. (2001) The white spot syndrome virus DNA genome sequence. Virology 286:7-22
van Hulten M C W, Goldbach R W, Vlak J M (2000) Three functionally diverged major structural proteins of white spot syndrome virus evolved by gene duplication. J Gen Virol 81:2525-2529
Yang F et al. (2001) Complete genome sequence of the shrimp white spot bacilliform virus. J Virol 75:11811-11820
Yao K, Vakharia V N (1998) Generation of infectious pancreatic necrosis virus from cloned cDNA. J Virol 72:8913-8920
Zhang X-K, Takashima I, Hashimoto N (1989) Characteristics of passive immunity against hantavirus infection in rats. Arch Virol 105:235-246

We claim:

1. A method of protecting an animal from disease, said method comprising:
a. producing a disease-related protein or peptide from the agent that causes the disease in a transformed host cell; and
b. delivering the protein or peptide to an animal suspected of being infected by the agent,
wherein delivery of the protein inhibits or retards binding of the agent that causes the disease in one or more cells of the animal.

2. The method of claim 1, wherein the agent that causes the disease is a virus.

3. The method in claim 1, wherein the agent that causes the disease is a bacterium.

4. The method in claim 1, wherein the agent that causes the disease is a prion.

5. The method of claim 1, wherein said producing comprises transforming a host cell with a nucleic acid encoding the disease-related protein to form a transformed cell.

6. The method of claim 1, wherein the host cell is chosen from bacteria, algae, yeast, fungi, insects, animals, plants, and tissue cultures of any of the above.

7. The method of claim 6, wherein the host cell is an alga.

8. The method of claim 6, wherein the host cell is a yeast.

9. The method of claim 6, wherein the host cell is a bacterium.

10. The method of claim 1, wherein the disease-related protein is a fusion protein.

11. The method of claim 1, wherein the disease-related protein is a viral protein.

12. The method of claim 11, wherein the viral protein comprises one or more segments of white spot syndrome virus.

13. The method of claim 12, wherein the viral protein comprises one or more segments of white spot syndrome viral protein VP26.

14. The method of claim 12, wherein the viral protein comprises one or more segments of white spot syndrome viral protein VP28.

15. The method of claim 12, wherein the viral protein comprises one or more segments of white spot syndrome viral protein VP19.

16. The method of claim 12, wherein the viral protein comprises one or more segments of white spot syndrome viral protein VP24.

17. A feed comprising a recombinant protein or peptide capable of binding to a disease-causing agent.

18. The feed of claim 17, wherein the recombinant protein or peptide comprises at least a portion of a viral protein.

19. The feed of claim 18, wherein the recombinant protein or peptide comprises white spot syndrome virus sequences.

20. The feed of claim 19, wherein the recombinant protein or peptide comprises sequences chosen from one or more of VP24, VP28, VP26, and VP19.

21. A feed additive comprising a recombinant protein or peptide capable of binding to a disease-causing agent.

22. The feed additive of claim 21, which is fed to an animal as whole cells or broken cells.

23. The feed additive of claim 21, which is fed to an animal as purified or semi-purified protein, or encapsulated versions of these.

24. The feed additive of claim 21, comprising a recombinant protein or peptide comprising at least a portion of a viral protein.

25. The feed additive of claim 24, wherein the recombinant protein or peptide comprises white spot syndrome virus sequences.

26. The feed additive of claim 25, wherein the recombinant protein or peptide comprises sequences from one or more of VP24, VP28, VP26, and VP19.

27. A method of protecting an animal from disease, said method comprising:
a. producing a protein or peptide capable of binding a disease-causing agent in a transformed host cell; and
b. delivering the protein or peptide to an animal suspected of being infected by the agent,
wherein delivery of the protein inhibits or retards binding of the agent that causes the disease in one or more cells of the animal.

28. A feed comprising a recombinant protein or peptide capable of binding a disease-causing agent and reducing or alleviating a disease state.

29. A feed additive comprising a protein or peptide capable of binding to a disease-causing agent.

30. The feed additive of claim 29, which is fed as whole cells or broken cells.

31. The feed additive of claim 30, which is fed as purified or semi-purified protein, or encapsulated versions of these.
